(19) United States
(12) Patent Application Publication
(10) Pub. No.: US 2024/0312172 A1
(43) Pub. Date: Sep. 19, 2024

(54) IMMEDIATE STITCHING AND RECOVERY STITCHING

(71) Applicant: Align Technology, Inc., San Jose, CA (US)

(72) Inventors: Shai Ayal, Tel Aviv (IL); Gal Peleg, Kiryat-Ono (IL); Eliran Dafna, Beer-sheva (IL); Sergei Ozerov, Yerevan (AM); Sergei Koshman, Yerevan (AM)

(21) Appl. No.: 18/607,297

(22) Filed: Mar. 15, 2024

Related U.S. Application Data
(60) Provisional application No. 63/452,916, filed on Mar. 17, 2023.

(51) Int. Cl. G06T 19/20; A61B 1/00; A61B 1/24; A61C 9/00; A61C 13/34

(52) U.S. Cl. CPC G06T 19/20; A61B 1/00172; A61B 1/24; A61C 9/006; A61C 13/34; G06T 2200/04; G06T 2200/08; G06T 2210/41; G06T 2219/2004; G06T 2219/2008; G06T 2219/2016

(57) ABSTRACT

A method is provided, including using at least one computer processor, for receiving, during intraoral scanning of an intraoral three-dimensional (3D) surface by an intraoral scanner, a plurality of intraoral scans of the intraoral 3D surface generated by the intraoral scanner, each intraoral scan including an image of a distribution of discrete unconnected spots of light projected on the intraoral 3D surface, registering the plurality of intraoral scans to each other during the intraoral scanning to update a 3D model of the intraoral 3D surface at a rate of 3-100 times per second, and outputting a view of the 3D model of the intraoral 3D surface to a display. Other applications are also described.

### CROSS-REFERENCE TO RELATED APPLICATIONS

The present application claims the priority of U.S. 63/452,916 to Ayal et al., filed Mar. 17, 2023, which is incorporated herein by reference.

### FIELD OF THE INVENTION

The present disclosure relates generally to three-dimensional imaging, and more particularly to intraoral three-dimensional imaging.

### BACKGROUND

Digital dental impressions utilize intraoral scanning to generate three-dimensional digital models of an intraoral three-dimensional surface of a subject. Digital intraoral scanners may use structured light three-dimensional imaging using a combination of structured light projectors and cameras disposed within the intraoral scanner.

US 2019/0388193 to Saphier et al., which is assigned to the assignee of the present application and is incorporated herein by reference, describes an apparatus for intraoral scanning including an elongate handheld wand that has a probe. One or more light projectors and two or more cameras are disposed within the probe. The light projectors each have a pattern generating optical element, which may use diffraction or refraction to form alight pattern. Each camera may be configured to focus between 1 mm and 30 mm from a lens that is farthest from the camera sensor. Other applications are also described.

US 2019/0388194 to Atiya et al., which is assigned to the assignee of the present application and is incorporated herein by reference, describes a handheld wand including a probe at a distal end of the elongate handheld wand. The probe includes a light projector and a light field camera. The light projector includes a light source and a pattern generator configured to generate a light pattern. The light field camera includes a light field camera sensor. The light field camera sensor includes (a) an image sensor including an array of sensor pixels and (b) an array of micro-lenses disposed in front of the image sensor such that each micro-lens is disposed over a sub-array of the array of sensor pixels. Other applications are also described.

US 2020/0404243 to Saphier et al., which is assigned to the assignee of the present application and is incorporated herein by reference, describes a method for generating a 3D image, including driving structured light projector(s) to project a pattern of light on an intraoral 3D surface, and driving camera(s) to capture images, each image including at least a portion of the projected pattern, each one of the camera(s) comprising an array of pixels. A processor compares a series of images captured by each camera and determines which of the portions of the projected pattern can be tracked across the images. The processor constructs a three-dimensional model of the intraoral three-dimensional surface based at least in part on the comparison of the series of images. Other embodiments are also described.

### SUMMARY OF THE INVENTION

Applications of the present disclosure include systems and methods relating to stitching together incoming scans from an intraoral scanner to generate a 3D image of an intraoral 3D surface being scanned. Typically, the intraoral scanner includes an elongate wand (e.g., an elongate handheld wand) with a probe at a distal end of the wand. The probe has a transparent window through which light rays enter and exit the probe. One or more cameras are disposed within the probe and arranged within the probe such that the one or more cameras receive rays of light from an intraoral cavity through the transparent window. One or more structured light projectors are disposed within the wand and arranged to project a pattern of structured light features onto the intraoral surface. One or more broadband light projectors are disposed within the handheld wand and arranged to illuminate the intraoral surface such that 2D images of the intraoral 3D surface under broadband illumination may be captured during the intraoral scanning.

Images captured by the one or more cameras of the projected pattern of structured light features on the intraoral 3D surface are used to generate a 3D image of the intraoral 3D surface being scanned. Typically, the images captured in each structured light image frame are used to compute a 3D point cloud of 3D positions of projected features of the pattern of light on the intraoral 3D surface, for example using techniques described in US 2019/0388193 to Saphier et al., US 2019/0388194 to Atiya et al., and US 2020/0404243 to Saphier et al. The computed point clouds from each structured light image frame are stitched together in real-time, e.g., soft real-time, during the intraoral scanning, as further described hereinbelow, in order to generate the 3D image of the intraoral 3D surface while the intraoral scan is ongoing.

In accordance with some applications of the present disclosure, a simultaneous localization and mapping (SLAM) algorithm is used to track motion of the wand and to generate 3D images. A SLAM algorithm typically iteratively determines the position of the intraoral scanner relative to scanned object (“localization”) then uses this position to update knowledge of scanned object (“mapping”). For some applications, one or more SLAM algorithms described in US 2020/0404243 to Saphier et al. may be used.

It is noted that registering 3D point clouds together refers to orienting and/or positioning the 3D point clouds relative to each other so that the 3D point clouds can be stitched together into a larger combined 3D point cloud. The use of the word “stitch” (in any form) throughout the present application is interchangeable with “register,” and refers to the process of registration and stitching of 3D point clouds together. Thus, for example, “stitching seed” as used hereinbelow is interchangeable with “registration seed” as used hereinbelow.

During scanning of an intraoral 3D surface, a processing device (e.g., a computer processor) receives a new “scan” for each structured light image frame. Thus, during intraoral scanning of the intraoral 3D surface, the processing device receives a plurality of consecutive intraoral scans of the intraoral 3D surface generated by the intraoral scanner. The processing device computes a 3D point cloud of 3D positions of projected features of the pattern of light on the intraoral 3D surface for each new scan. The 3D point clouds are stitched to each other and displayed on a user display so that the user of the intraoral scanner sees the 3D image being generated during the scanning. Typically, the processor works to stitch the incoming 3D point clouds to each other in soft real-time (further described hereinbelow). For example, the processor may update the 3D image on the display 3-100 times per second, e.g., 10-60 times per second, e.g., 40-60 times per second.

Typically, the features of the projected pattern in each structured light image frame are sparse, which, in turn results in a sparse 3D point cloud for each intraoral scan. Typically, each intraoral scan has at least 100 and/or less than 1000 3D points in the corresponding 3D point cloud. The sparsity of the 3D point clouds presents challenges in registering the 3D point clouds to each other. Thus, for some applications, instead of registering each 3D point cloud to the 3D point cloud corresponding to the previous intraoral scan, each incoming scan of a subset of the plurality of intraoral scans received during the intraoral scanning is registered to a registration seed (also referred to hereinbelow as “stitching seed”). The registration seed is a subset of the intraoral scans that immediately precede the incoming scan and that have previously been stitched together. That is, a 3D point cloud corresponding to an incoming intraoral scan is registered to a larger 3D point cloud containing 3D points from a set of intraoral scans, thus increasing the amount of information available for the registration. It is noted that as used throughout this application including in the claims, a “subset” is a set each of whose elements is an element of an inclusive set. Thus, a subset includes the original set or any smaller grouping of the elements in the original set.

There is therefore provided, in accordance with some applications of the present invention, a method including, using at least one computer processor:
receiving, during intraoral scanning of an intraoral three-dimensional (3D) surface by an intraoral scanner, a plurality of intraoral scans of the intraoral 3D surface generated by the intraoral scanner, each intraoral scan comprising an image of a distribution of discrete unconnected spots of light projected on the intraoral 3D surface;
registering the plurality of intraoral scans to each other during the intraoral scanning to update a 3D model of the intraoral 3D surface, wherein the 3D model is updated at a rate of 3-100 times per second; and
outputting a view of the 3D model of the intraoral 3D surface to a display.

For some applications, each intraoral scan includes an image of a distribution of 400-3000 discrete unconnected spots of light projected on the intraoral 3D surface.

There is further provided, in accordance with some applications of the present invention, a method including, using at least one computer processor:
receiving, during intraoral scanning of an intraoral three-dimensional (3D) surface by an intraoral scanner, a plurality of consecutive intraoral scans of the intraoral 3D surface generated by the intraoral scanner, each intraoral scan comprising an image of a distribution of discrete unconnected spots of light projected on the intraoral 3D surface;
registering the plurality of intraoral scans to each other during the intraoral scanning to update a segment of a 3D model of the intraoral 3D surface by registering each incoming scan of a subset of the plurality of intraoral scans to a registration seed, wherein the registration seed is a subset of intraoral scans that immediately precede the incoming scan and that have previously been registered together; and outputting a view of the segment of the 3D model of the intraoral 3D surface to a display.

For some applications, each intraoral scan includes an image of a distribution of 400-3000 discrete unconnected spots of light projected on the intraoral 3D surface.

For some applications, the segment of the 3D model is updated at a rate of 3-100 times per second.

For some applications, the registration seed includes a subset of the last 2-8 intraoral scans generated by the intraoral scanner during the intraoral scanning.

For some applications, the method further includes, using the at least one computer processor, selecting a quantity of intraoral scans to be included in the registration seed such that the registration seed includes images of a combined total of 500-5000 discrete unconnected spots of light projected on the intraoral 3D surface.

For some applications:
the method further includes, using the at least one computer processor, computing a source 3D point cloud from the distribution of discrete unconnected spots of light captured in each incoming scan and computing a target 3D point cloud from the registration seed,
registering each incoming scan of the subset of the plurality of intraoral scans to the registration seed includes using an Iterative Closest Point (ICP) algorithm to register the source 3D point cloud to the target 3D point cloud, and
the method further includes, using the at least one computer processor:
   receiving, during the intraoral scanning, a plurality of 2D images of the intraoral 3D surface captured under broadband illumination,
   pairing one or more of the plurality of 2D images with the incoming scan, and based on the pairing, classifying a subset of the points in the source 3D point cloud as corresponding to a spot of light projected on rigid tissue, and
   using as an input to the ICP algorithm only the points in the source 3D point cloud that are classified as corresponding to a spot of light projected on rigid tissue.

For some applications:
the method further includes, using the at least one computer processor, computing a source 3D point cloud from the distribution of discrete unconnected spots of light captured in each incoming scan and computing a target 3D point cloud from the registration seed,
registering each incoming scan of the subset of the plurality of intraoral scans to the registration seed includes using an Iterative Closest Point (ICP) algorithm to register the source 3D point cloud to the target 3D point cloud, and
the method further includes, using the at least one computer processor:
   receiving, during the intraoral scanning, data pertaining to motion of the intraoral scanner from an inertial measurement unit (IMU) disposed within the intraoral scanner,
   for each incoming scan of the subset of the plurality of intraoral scans, using a Kalman filter to predict an expected position of the intraoral scanner by extrapolating a previous trajectory of the motion of the intraoral scanner using the data from the IMU, and
   providing the ICP algorithm with an initial estimate based on (i) the expected position predicted by the Kalman filter, and (ii) the intraoral scan that immediately preceded the incoming scan.

For some applications:
the method further includes, using the at least one computer processor, computing a source 3D point cloud from the distribution of discrete unconnected spots of light captured in each incoming scan and computing a target 3D point cloud from the registration seed,
registering each incoming scan of the subset of the plurality of intraoral scans to the registration seed includes using an Iterative Closest Point (ICP) algorithm to register the source 3D point cloud to the target 3D point cloud, and
the method further includes, using the at least one computer processor:
   attempting to compute a normal for each point in the source 3D point cloud, and
   using as an input to the ICP algorithm only the points in the source 3D point cloud for which the normal was successfully computed.

For some applications:
the method further includes, using the at least one computer processor, computing a source 3D point cloud from the distribution of discrete unconnected spots of light captured in each incoming scan and computing a target 3D point cloud from the registration seed,
registering each incoming scan of the subset of the plurality of intraoral scans to the registration seed includes using an Iterative Closest Point (ICP) algorithm to register the source 3D point cloud to the target 3D point cloud, and
the method further includes, using the at least one computer processor, subsequently to the ICP algorithm registering the source 3D point cloud to the target 3D point cloud, assessing an extent of overlap between the source 3D point cloud and the target 3D point cloud.

For some applications, assessing the extent of overlap includes:
outputting a subset of pairs of matching points between the source 3D point cloud and the target 3D point cloud, each pair of points determined to be matching points by the ICP algorithm,
for each pair of matching points, extracting a plurality of features,
inputting the extracted features into a classifier algorithm,
receiving from the classifier algorithm an overlap-grading-score relating to the extent of overlap between the source 3D point cloud and the target 3D point cloud, and
rejecting the registration of the incoming scan by the ICP algorithm in response to the overlap-grading-score being lower than a threshold score.

For some applications:
the method further includes, using the at least one computer processor, receiving, during the intraoral scanning, a plurality of 2D images of the intraoral 3D surface captured under broadband illumination, and
extracting a plurality of features includes extracting a plurality of features wherein at least one of the extracted features is a non-geometrical feature based on the plurality of 2D images of the intraoral 3D surface captured during capturing of the incoming scan.

For some applications, extracting the plurality of features includes extracting 2-50 features.

For some applications, inputting the extracted features into a classifier algorithm includes inputting the extracted features into a neural network.

For some applications, inputting the extracted features into a classifier algorithm includes inputting the extracted features into a support vector machine.

For some applications:
the method further includes, using the at least one computer processor, computing a source 3D point cloud from the distribution of discrete unconnected spots of light captured in each incoming scan and computing a target 3D point cloud from the registration seed,
registering each incoming scan of the subset of the plurality of intraoral scans to the registration seed includes using an Iterative Closest Point (ICP) algorithm to register the source 3D point cloud to the target 3D point cloud, and
the method further includes, using the at least one computer processor:
   receiving, during the intraoral scanning, data pertaining to motion of the intraoral scanner from an inertial measurement unit (IMU) disposed within the intraoral scanner,
   assessing the registration by the ICP algorithm of the source 3D point cloud to the target 3D point cloud by comparing the registration to data from the IMU, and
   rejecting the registration of the incoming scan by the ICP algorithm in response to the registration of the source 3D point cloud to the target 3D point cloud contradicting the data from the IMU.

For some applications, the method further includes, using the at least one computer processor:
when no registration seed is available:
   (a) receiving a first incoming scan by the intraoral scanner,
   (b) receiving a second incoming scan by the intraoral scanner,
   (c) attempting to register the second incoming scan to the first incoming scan,
   (d):
      (i) if the attempted registration of the second incoming scan to the first incoming scan is successful, storing the set of the first and second incoming scans that have been registered to each other as a candidate set for the registration seed, and
      (ii) if the attempted registration of the second incoming scan to the first incoming scan is unsuccessful, storing each of the first and second incoming scans separately as candidate sets for the registration seed,
   (e) receiving a next incoming scan by the intraoral scanner and attempting to register the next incoming scan to each of the stored candidate sets generated in step (d),
   (f):
      (i) if the attempted registration of the next incoming scan to a candidate set is successful, updating the candidate set, and
      (ii) if no attempted registration of the next incoming scan to a candidate set is successful, storing the next incoming scan as a new candidate set for the registration seed, and
   (g) iteratively repeating steps (e) and (f) until at least one candidate set is large enough to be used as the registration seed.

For some applications, the method further includes, using the at least one computer processor, discarding the oldest candidate set when the number of stored candidate sets surpasses a threshold number.

For some applications, the threshold number is between 2 and 10.

For some applications, the method further includes, using the at least one computer processor:
(a) receiving a first incoming scan by the intraoral scanner and attempting to register the first incoming scan to the registration seed,
(b) if the registration of the first incoming scan to the registration seed fails, storing the first incoming scan as a scan in a backup candidate set of one or more intraoral scans,
(c) receiving a second incoming scan by the intraoral scanner and attempting to register the second incoming scan to registration seed, and
(d):
   (i) if the registration of the second incoming scan to the registration seed fails, attempting to register the second incoming scan to the backup candidate set,
      (1) if the attempted registration to the backup candidate set is successful, updating the backup candidate set, and
      (2) if the attempted registration to the backup candidate set is unsuccessful, storing each of the first incoming scan and the second incoming scan separately, as scans in respective first and second backup candidate sets of one or more intraoral scans, and
   (ii) if the registration of the second incoming scan to the registration seed is successful, discarding the backup candidate sets generated in step (b).

For some applications, the method further includes, using the at least one computer processor:
(e) receiving a next incoming intraoral scan and attempting to register the next incoming intraoral scan to the registration seed, and
(f):
   (i) if the registration of the next incoming scan to the registration seed fails, attempting to register the second incoming scan to each of the backup candidate sets generated in step (d):
      (1) if the attempted registration of the next incoming scan to a backup candidate set is successful, updating the backup candidate set,
      (2) if no attempted registration of the next incoming scan to a backup candidate set is successful, storing the next incoming scan as a scan in a new backup candidate set of one or more intraoral scans, and
      (3) if no backup candidate sets were stored in step (d), storing the next incoming scan as a scan in a new backup candidate set of one or more intraoral scans, and
   (ii) if the registration of the next incoming scan to the registration seed is successful, discarding all stored backup candidate sets.

For some applications, the segment of the 3D model is a main segment of the 3D model, and the method further includes, using the at least one computer processor:
(g) iteratively repeating steps (e) and (f) until a given backup candidate set grows large enough to be considered a backup segment of the 3D model of the intraoral surface,
(h) receiving a next incoming intraoral scan and attempting to register the next incoming intraoral scan to the registration seed of the main segment, and
(i):
   (A) if the registration of the next incoming scan to the registration seed of the main segment fails, attempting to register the next incoming scan to a backup registration seed of the backup segment, wherein the backup registration seed is a subset of intraoral scans in the backup segment that immediately precede each next incoming scan, wherein:
      (1) if the attempted registration of the next incoming scan to the backup registration seed is successful, updating the backup segment of the 3D model, and
      (2) (a) if the attempted registration of the next incoming scan to the backup registration seed fails, increasing a counter of failed attempts to register a next incoming scan to the backup registration seed, and (b) if the counter exceeds a threshold number, discarding the backup segment and storing the next incoming scan as a scan in a new backup candidate set of one or more intraoral scans, and
   (B) if the registration of the next incoming scan to the registration seed of the main segment is successful, discarding the backup segment.

For some applications, iteratively repeating steps (e) and (f) until a given backup candidate set grows large enough to be considered a backup segment of the 3D model of the intraoral surface further includes attempting to register the backup segment of the 3D model to the main segment of the 3D model.

For some applications:
(I) if the attempted registration of the backup segment of the 3D model to the main segment of the 3D model is successful, merging the backup segment of the 3D model with the main segment of the 3D model, outputting a view of the merged 3D model of the intraoral surface to the display, and subsequently using the backup registration seed of the backup segment as the registration seed of the merged 3D model for future income scans, and
(II) if the attempted registration of the backup segment of the 3D model to the main segment of the 3D model fails, waiting for the earlier of: (1) the backup segment to be updated, in which case the method further comprises attempting to register the updated backup segment to the main segment, or (2) a new backup candidate set of one or more intraoral scans to grow large enough to be considered a new backup segment, in which case the method further comprises attempting to register the new backup segment to the main segment.

For some applications, if upon performing step II the attempted registration of the updated backup segment to the main segment or of the new backup segment to the main segment fails, the method further comprises iteratively repeating the waiting of step (II), wherein if no registration of any backup segment to the main segment is successful for a threshold amount of time, the method further includes storing the main segment of the 3D model as a first part of the 3D model of the intraoral surface and using the last backup segment stored as the beginning of a new main segment for a second part of the 3D model of the intraoral surface.

For some applications, attempting to register the backup segment of the 3D model to the main segment of the 3D model comprises using a Kalman filter algorithm.

For some applications, using a Kalman filter algorithm includes:
computing a source 3D point cloud from the backup segment of the 3D model and computing a target 3D point cloud from the main segment of the 3D model,
splitting the target 3D point cloud into a plurality of sections, and
using a modified Iterative Closest Point (ICP) algorithm to attempt to register the source 3D point cloud computed from the backup segment to a plurality of the plurality of sections of the target 3D point cloud using a plurality of iterations, wherein for each iteration of the modified ICP algorithm a position of the target 3D point cloud is modified, wherein position and rotation of the target 3D point cloud are defined by a state of the Kalman filter.

For some applications, the method further includes, using the at least one computer processor, receiving, during the intraoral scanning, a plurality of 2D images of the intraoral 3D surface captured under broadband illumination, and wherein using the Kalman filter algorithm further includes, using the at least one computer processor:
looking for 2D images of the intraoral 3D surface corresponding to the backup segment that are similar to 2D images of the intraoral 3D surface corresponding to the main segment, and
using the similarity between the 2D images as an initial guess for the modified ICP algorithm.

The present invention will be more fully understood from the following detailed description of applications thereof, taken together with the drawings, in which:

### BRIEF DESCRIPTION OF THE DRAWINGS

FIG. 1 depicts an immediate stitching algorithm used by a processing device, in accordance with some applications of the present invention; and

FIGS. 2-4 each depict a respective component of the immediate stitching algorithm in more detail, in accordance with some applications of the present invention.

### DETAILED DESCRIPTION

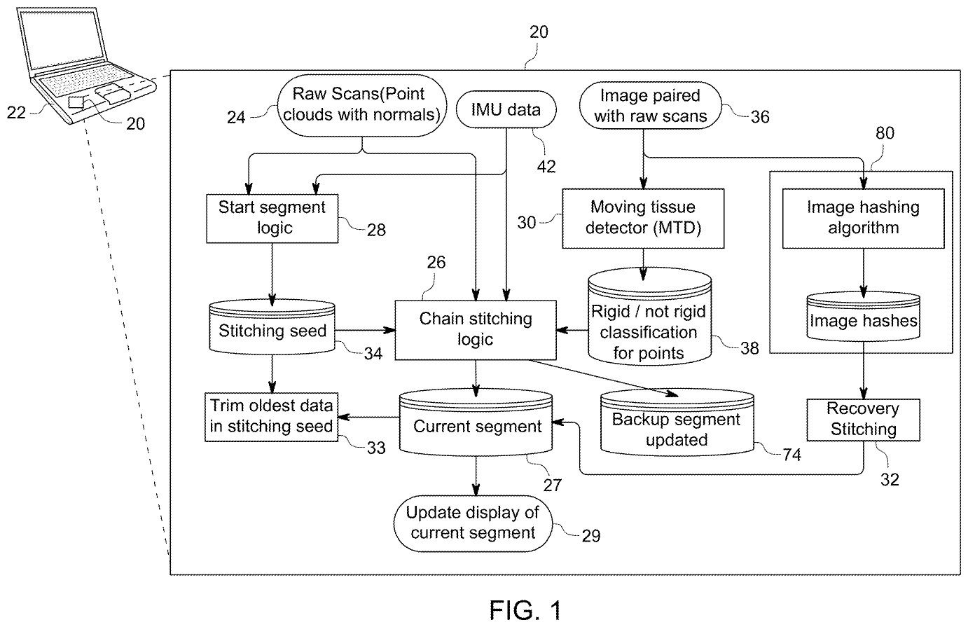

Reference is now made to FIG. 1, which depicts an immediate stitching algorithm 20 used by a processing device 22 of an intraoral scanning system comprising the processing device and an intraoral scanner, in accordance with some applications of the present invention. The processing device may be, e.g., at least one computer processor, in accordance with some applications of the present invention. Typically, during intraoral scanning of the intraoral 3D surface by an intraoral scanner of an intraoral scanning system, processing device 22 receives a plurality of consecutive intraoral scans (step 24) of the intraoral 3D surface generated by the intraoral scanner.

For some applications, the field of view of the intraoral scanner is wide. For example, the field of view of each of the cameras may be at least 45 degrees, e.g., at least 80 degrees, e.g., 85 degrees. Optionally, the field of view of each of the cameras may be less than 120 degrees, e.g., less than 90 degrees. In addition, for some applications, the number of features of the projected pattern in each structured light image frame is sparse, e.g., each intraoral scan includes an image of a distribution of discrete unconnected spots of light projected on the intraoral 3D surface. For example, each structured light projector may project at least 400 and/or less than 3000 features, e.g., spots of light. Both the wide field of view of the intraoral scanner and the sparsity of the projected features present challenges to the stitching of the 3D point clouds, in particular to the stitching of 3D point clouds from structured light image frames that are early on in an intraoral scan.

Some additional challenges are as follows:
Since each incoming scan is stitched to the scans that proceeded it, an error in the stitching of one scan can result in future scans being positioned incorrectly, this is known as a “stitching artifact.” As further described hereinbelow, the inventors have realized a grading method used to increase the accuracy of the stitching.
Some parts of the intraoral cavity may move during a scan, e.g., the tongue, cheeks, the fingers of the dentist performing the intraoral scan, and tools in the intraoral cavity. This can result in stitching errors. As further described hereinbelow, the inventors have realized a way for the intraoral scanner to distinguish between generally rigid parts of the intraoral cavity (e.g., teeth and gums) and moving tissues (e.g., tongue, check, fingers) such that only parts of each scan corresponding to rigid tissue are for stitching.
A premise that is relied upon for the SLAM algorithm is that since the mapping is updated 40-60 times per second, and the movement of the intraoral scanner with respect to the intraoral 3D surface being scanned is typically slower than 40 mm/s, it can be assumed that for each scan the position of the intraoral scanner is very close to its position in the previous scan. However, at times there may be not enough of an object in the field of view of the intraoral scanner for the position of the intraoral scanner to be determined. If this happens for a sufficient period of time, then the assumption that each scan is close in position to the previous scan can no longer be relied upon. As further described hereinbelow, the inventors have realized a “recovery stitching” algorithm to address this.

The overall immediate stitching algorithm 20 is used by processing device 22 to build the 3D image of the intraoral 3D surface while the 3D surface is being scanned, i.e., registering the plurality of intraoral scans to each other (step 26, also referred to herein as “chain stitching”) during the intraoral scanning to update a segment 27 of a 3D model of the intraoral 3D surface and outputting a view of the 3D model, e.g., of a segment of the 3D model, of the intraoral surface to a display (step 29). Immediate stitching algorithm 20 includes multiple components. The stitching of each incoming scan to a set of previously stitched scans is referred to herein as “chain stitching” (step 26). The chain stitching component (step 26) of immediate stitching algorithm 20 handles most of the incoming scans and attempts to stitch each new scan to part of an existing “segment,” i.e., set of previously stitched scans. As described further hereinbelow, there are also dedicated components in the stitching algorithm for (i) starting a new segment (step 28), (ii) detecting and excluding moving tissue from consideration (step 30), and (iii) handling cases where the chain stitching fails to stich an incoming scan to an existing segment, referred to herein as “recovery stitching” (step 32).

Typically, the processor works to stitch the incoming 3D point cloud to the existing segment in soft real-time such that there are enough updates per second to the 3D image being shown on the user display and low enough latency (i.e., the time between when a new structured light image frame is captured and when the user sees the corresponding update to the 3D image on the user display) so that the user of the intraoral scanner experiences a real-time effect of the 3D image being generated on the user display as the scan is ongoing. For example, the processor may update the 3D image on the display 3-100 times per second, e.g., 10-60 times per second, e.g., 40-60 times per second. Thus, for some applications, the latency time is less than 300 milliseconds (ms), e.g., less than 200 ms, e.g., less than 150 ms, e.g., less than 100 ms, e.g., less than 50 ms, e.g., less than 15 ms.

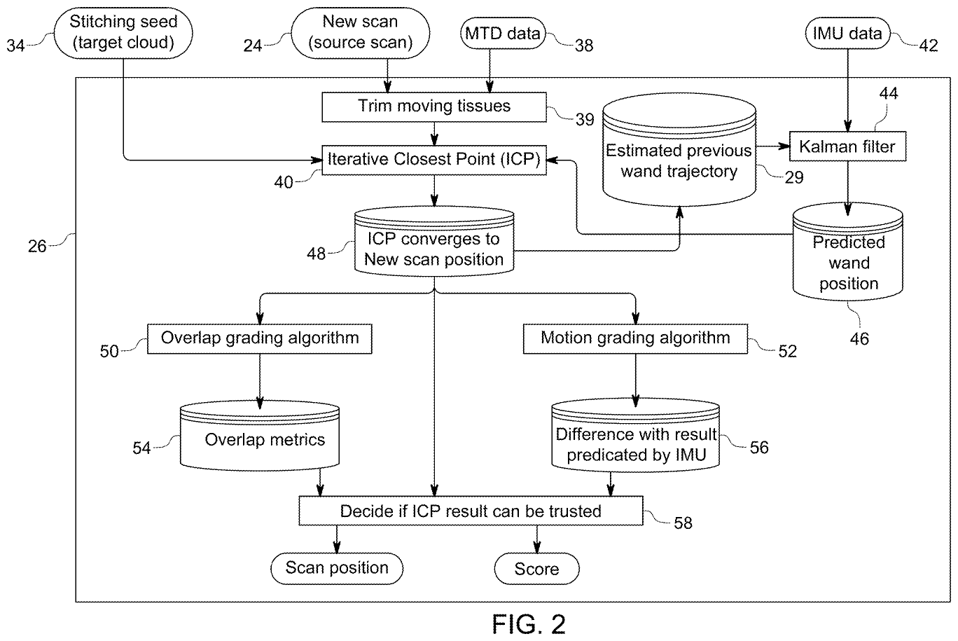

Chain stitching component (step 26) of immediate stitching algorithm 20 is based on Iterative Closest Point (ICP) stitching between point clouds, in which a source cloud is stitched to a target cloud. The segment to which the incoming scans are stitched is a target cloud that grows linearly as scans are added to it. Each incoming intraoral scan is a source cloud. As described hereinabove, the source cloud for each incoming scan is sparse, thus presenting challenges in stitching each incoming scan to the single scan last stitched to the segment. Thus, each incoming scan is stitched to a plurality of previously stitched scans. For some applications, chain stitching component 26 registers each incoming scan of a subset of the plurality of intraoral scans received during the intraoral scanning to a subset of the intraoral scans that immediately precede the incoming scan and that have been previously registered together. The subset of the intraoral scans that immediately precede the incoming scan, to which the incoming scan is registered, is referred to herein as a “stitching seed” 34 (used herein interchangeably with “registration seed”). Thus, for some applications, processing device 22 (i) computes a source 3D point cloud from the distribution of discrete unconnected features, e.g., spots, of light captured in each incoming scan and (ii) computes a target 3D point cloud from the registration seed. The ICP algorithm (step 40, shown in FIG. 2) of the chain stitching component (step 26) registers the source 3D point cloud to the target 3D point cloud.

In order to save processing time, once a segment is large enough, only a subset of points in the segment, e.g., a subset consisting of recent points added to the target cloud, are used as the stitching seed. For example, the registration seed may include a subset of the last 2-8 intraoral scans generated by the intraoral scanner during the intraoral scanning. For example, each incoming scan may be stitched to the segment using only the last few thousand points, e.g., at least 500 points and/or less than 5000 points, added to the target cloud. Thus, for some applications, processing device 22 selects a quantity of intraoral scans to be included in the registration seed such that the registration seed includes images of a combined total of at least 500 and/or less than 5000 discrete unconnected features, e.g., spots, of light projected on the intraoral 3D surface. For some applications, the ICP stitching takes less than 10 ms, e.g., less than 5 ms, e.g., 4 ms to converge. Incoming intraoral scans that are added to current segment 27 are added to the registration seed of current segment 27. Thus, as more incoming scans are added, processing device 22 trims the data stored in the registration seed (step 33), i.e., trims the number of scans included in the registration seed, thus updating registration seed 34.

For some applications, the chain stitching component of the immediate stitching algorithm combines ICP with a moving tissue detection (MTD) algorithm (step 30). Typically, in addition to the structured light projectors, there is also one or more non-structured light projectors which project broadband light, e.g., white light, into the intraoral cavity. The one or more cameras capture 2D images under the broadband illumination from the non-structured light projectors. The MTD algorithm pairs the 2D images with raw scan data (step 36) in order to determine which parts of the scan include moving tissue. For each incoming scan the points in the source cloud are given a rigid or non-rigid classification based on the MTD algorithm (step 38). Typically, only the points classified as rigid are used for the ICP stitching to the target cloud.

Thus, for some applications, processing device 22 receives, during the intraoral scanning, a plurality of 2D images of the intraoral 3D surface captured under broadband illumination and pairs one or more of the plurality of 2D images with each incoming scan (step 36). Based on the pairing, in step 38 processing device 22 classifies a subset of the points in the source 3D point cloud (computed from the incoming scan) as corresponding to a feature, e.g., spot, of light projected on rigid tissue (e.g., teeth or gums). Processing device 22 uses as an input to the ICP algorithm only the points in the source 3D point cloud that are classified as corresponding to a feature, e.g., spot, of light projected on rigid tissue (step 39, shown in FIG. 2). Additionally or alternatively, for some applications processing device 22 may attempt to compute a normal for each point in the source 3D point cloud (included in step 24), and use as an input to the ICP algorithm only the points in the source 3D point cloud for which the normal was successfully computed. Filtering the points this way helps to eliminate points which do not correspond to a spot of light projected onto the intraoral surface.

Reference is now made to FIG. 2, which depicts chain stitching logic 26 in more detail, in accordance with some applications of the present invention. For some applications, processing device 22 receives during the intraoral scanning, data pertaining to motion of the intraoral scanner from an inertial measurement unit (IMU) disposed within the intraoral scanner (step 42). The ICP stitching algorithm (step 40) typically pairs with a Kalman filter (step 44) that predicts the expected position of the intraoral scanner (step 46) by extrapolating the previous trajectory of the motion of the intraoral scanner using the data from the IMU. For some applications, the ICP stitching of a new scan may start with an initial estimate based on input from the IMU as well as the immediately preceding stitched scan. Thus, for some applications, for each incoming scan, processing device 22 (i) uses a Kalman filter to predict an expected position of the intraoral scanner by extrapolating a previous trajectory of the motion of the intraoral scanner using the data from the IMU, and (ii) provides the ICP algorithm (step 40) with an initial estimate based on (a) the expected position predicted by the Kalman filter in step 46 and (b) the intraoral scan that immediately preceded the incoming scan. The ICP stitching (step 40) typically converges to provide a position of the new scan (i.e., the incoming scan) with respect to the existing segment of the 3D model (step 48).

It is noted that use of a Kalman filter to predict the expected position of the intraoral scanner based on data from the IMU is byway of example and is not intended to be limiting. For some applications, other methods of predicting the expected position of the intraoral scanner based on data from the IMU may be utilized, e.g., integration of IMU samples. It is also noted that the expected position of the intraoral scanner may also be predicted without the use of data from an IMU. For example, for some applications, the expected position of the intraoral scanner may be predicted using the previous motion trajectory of the intraoral scanner without the use of an IMU, e.g., by interpolation and extrapolation of the motion of the intraoral scanner using Lie algebra and exponentiation.

Once the ICP stitching (step 40) converges and the new scan is added to the segment, the position of the intraoral scanner is typically added to the previous trajectory input to the Kalman filter in anticipation of the next scan. Most of the time the ICP converges to a fairly accurate estimate of the actual scan position within the 3D model of the intraoral 3D surface. However, sometimes it may converge to a wrong answer. As described hereinabove, an error in the stitching of one scan may propagate into a model-breaking stitching artifact. In order to avoid this, the ICP stitching is further paired with two algorithms that independently validate its output, referred to herein as “overlap grading” (step 50) and “motion grading” (step 52).

The overlap grading algorithm (step 50) attempts to estimate the overlap between each new incoming scan and the seed of the current segment to which it was stitched. That is, for some applications, subsequently to the ICP algorithm (step 40) registering the source 3D point cloud (computed from the incoming scan) to the target 3D point cloud (computed from the registration seed), processing device 22 assesses an extent of overlap between the source 3D point cloud and the target 3D point cloud (step 50). If the new scan is correctly stitched to the seed, then it is expected that at least a significant part of the new incoming scan and the seed should match almost exactly. The purpose of overlap grading is to “grade” the stitching quality of two point-clouds, namely the new incoming scan and the seed of the current segment. The overlap between the new incoming scan and the seed of the current segment (essentially the overlap between the surfaces scanned in consecutive structured light image frames) is affected by the motion speed of the intraoral scanner as the dentist or dental technician is moving it around the intraoral cavity. As a result of this, it is possible that there may be low overlap between consecutive scans. In order to allow fluent stitching between the point clouds the inventors have developed the overlap grading algorithm to enable stitching between the point clouds even if the overlap is small, as described hereinbelow.

For some applications, the overlap grading algorithm extracts features from two clouds, namely 3D point clouds of the new scan (source 3D point cloud) and the seed of the current segment (target 3D point cloud). After the ICP stitching attempts to stitch between the new scan and the seed of the current segment, a sub-set of matching points between the new scan and the seed of the current segment is produced. Using the pairs of matching points, the processing device extracts several features (step 54). As described hereinabove, for some applications, processing device 22 receives, during the intraoral scanning, a plurality of 2D images of the intraoral 3D surface captured under broadband illumination. Thus, for some applications, for each pair of matching points, at least one of the extracted features is a non-geometrical feature based on the plurality of 2D images of the intraoral 3D surface captured during capturing of the incoming scan.

For example, one or more of the extracted features may be from the following list:
Distances (Euclidian) between pairs
Normal similarities between pairs (each point is associated with a local surface normal)
Curvature similarities between pairs
Normal spreads (this measures how much “constraint” there is for “sliding” between surfaces)
Image similarity—each point is associated with an RGB color, a score from the MTD algorithm, and a “Distance-to-teeth” measure.

For some applications, at least 2 and/or less than 50, e.g., at least 5, e.g., at least 20, e.g., at least 40, e.g., 42 features are extracted. The extracted features are then fed into a classifier algorithm, e.g., a neural network or a support vector machine, that analyzes the inputs and then outputs a single overlap-grading-score ranging between [−1, 1], with zero being valued as a neutral decision. If the overlap-grading-score is lower than a threshold then processing device 22 rejects the registration of the incoming scan by the ICP algorithm. Thus, a stitched scan is considered to have passed the overlap grading if the score is above the threshold.

As described hereinabove, for some applications, processing device 22 receives, during the intraoral scanning, data pertaining to motion of the intraoral scanner from an inertial measurement unit (IMU) disposed within the intraoral scanner (step 42). The motion grading algorithm (step 52) validates the results (step 48) of the ICP stitching by comparing it against data from the IMU (step 56). Thus, for some applications, processing device 22 (i) assesses the registration by the ICP algorithm of the source 3D point cloud to the target 3D point cloud by comparing the registration to data from the IMU, and (ii) rejects the registration of the incoming scan by the ICP algorithm in response to the registration of the source 3D point cloud to the target 3D point cloud contradicting the data from the IMU. For some applications, the inertia of the moving intraoral scanner (based on the previous movement of the scanner) may also be used in the motion grading algorithm.

If the position of a stitched scan indicates that the intraoral scanner moved in a way that contradicts the scanner's IMU data, then the stitching of that scan is considered to have failed the motion grading validator. For example, a dentist performing an intraoral scan cannot instantly reverse the direction of motion of the intraoral scanner, thus if the position of the stitched scan indicates an instant reversal of direction of the intraoral scanner, then the stitching of that scan is considered to have failed the motion grading validator. The motion grading decision is computed by statistical learning, together with some of the metrics from the overlap grading algorithm. For some applications, motion metrics include shift and angular decline from the last motion position of the intraoral scanner. For some applications, some of the motion metrics compare (i) the projected trajectory of the intraoral scanner from its last position, to (ii) the position where the new scan actually stitched to the seed of the current segment. Additionally, a scanner motion speed limit may be used to reject scanning movements that are too fast.

For some applications a stitched scan must be accepted by the overlap grading and motion grading validators in order to pass further and become part of a segment (step 58). For some applications, if the motion of the intraoral scanner is smooth enough relative to the previous motion of the intraoral scanner, then the overlap grading may be skipped.

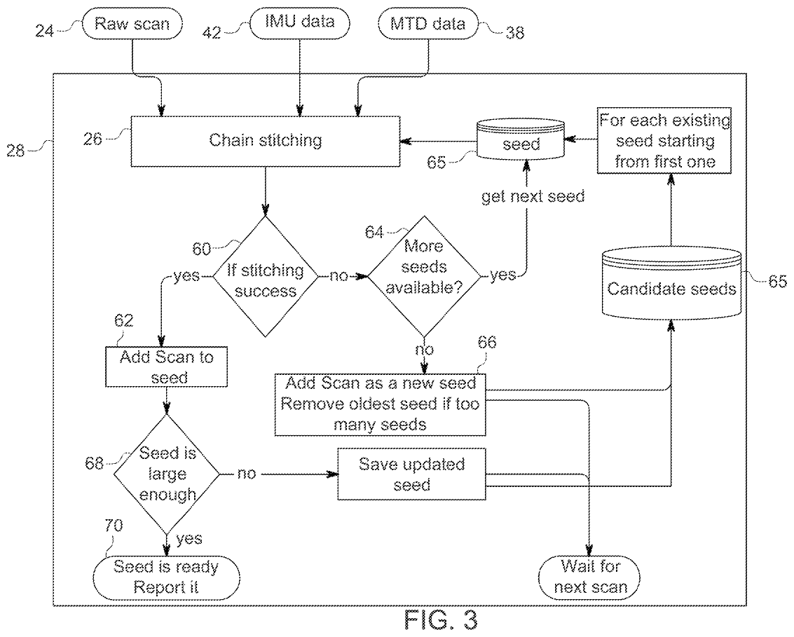

Reference is now made to FIG. 3, which depicts start segment logic 28 in more detail, in accordance with some applications of the present invention. As described hereinabove, the chain stitching needs a stitching seed to which to stitch each incoming scan. A simple algorithm for starting a new segment includes taking the first scan as the stitching seed and simply using the chain stitching to stich incoming scans to the seed. However, it may be the case that the incoming scans fail to stich to that first scan. The start-segment component of the immediate stitching algorithm handles this by keeping track of a plurality of seeds in parallel in order to see which one produces the most successful start to a segment.

For some applications, when no registration seed 34 is available, a plurality of scans are tracked as candidate seeds 65, e.g., up to a threshold number of initial scans. For some applications, the threshold may be between 2 and 10, e.g., up to 5 initial scans are tracked as candidate seeds. Subsequently, each new incoming scan (step 24), along with its corresponding IMU data (step 42) and MTD data (step 38), is inputted to the chain stitching component (step 26) of the immediate stitching algorithm to try and stitch the incoming scan to one of candidate seeds 65. If the stitching is successful (decision diamond 60) then that scan is added to the candidate seed 65 (step 62) to which it was stitched and that particular candidate seed “grows.” If the stitching is not successful (decision diamond 60) then the chain stitching component of the immediate stitching algorithm attempts to stitch the scan to the next available candidate seed 65. Decision diamond 64 indicates that if there is another available candidate seed 65 then the chain stitching component attempts to stitch the scan to the next available candidate seed and if there is no other available candidate seed 65 then the scan itself is added as a new candidate seed 65. That is, if the scan is not successfully stitched to any of the current candidate seeds being tracked then that scan itself becomes an additional candidate seed. If at any point there are too many candidate seeds, then older candidate seeds may be removed as newer ones are added in order to save processing time and space in memory. For some applications, once there are too many candidate seeds, instead of simply removing the oldest candidate seed each time a new candidate seed is added, processing device 22 may selectively remove the candidate seed that has the lowest number of data points. This process is repeated for each incoming new scan until one of the candidate seeds grows large enough to be considered the start of a new segment (decision diamond 68). At that point the other candidate seeds are discarded. The seed that grew large enough to be considered the start of a new segment is reported as the start of a new segment (step 70) and subsequent incoming scans are stitched to the seed of the new segment as described hereinabove.

An example workflow of start segment logic (step 28), performed by processing device 22, is as follows:
(a) receiving a first incoming scan by the intraoral scanner (including steps 24, 42, and 38),
(b) receiving a second incoming scan by the intraoral scanner (including steps 24, 42, and 38),
(c) attempting to register the second incoming scan to the first incoming scan (step 26),
(d):
   (i) if the attempted registration of the second incoming scan to the first incoming scan is successful (answer to decision diamond 60 is “yes”), storing the set of the first and second incoming scans that have been registered to each other as a candidate set for the registration seed (step 62 in combination with the answer to decision diamond 68 being “no”), and
   (ii) if the attempted registration of the second incoming scan to the first incoming scan is unsuccessful (answer to decision diamond 60 is “no”), storing each of the first and second incoming scans separately as candidate sets for the registration seed (step 66),
(e) receiving a next incoming scan by the intraoral scanner (including steps 24, 42, and 38) and attempting to register the next incoming scan (step 26) to each of the stored candidate sets generated in step (d),
(f):
   (i) if the attempted registration of the next incoming scan to a candidate set is successful (answer to decision diamond 60 is “yes), updating the candidate set (step 62), and
   (ii) if no attempted registration of the next incoming scan to a candidate set is successful (answer to decision diamonds 60 and 64 are both “no”), storing the next incoming scan as a new candidate set for the registration seed (step 66), and
(g) iteratively repeating steps (e) and (f) until at least one candidate set is large enough to be used as the registration seed (answer to decision diamond 68 is “yes”).

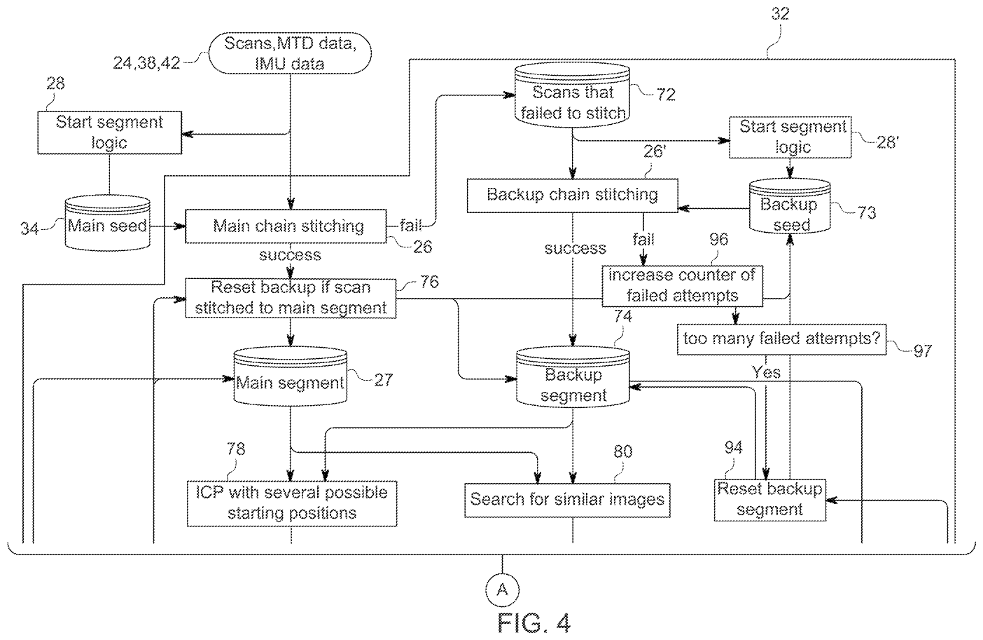

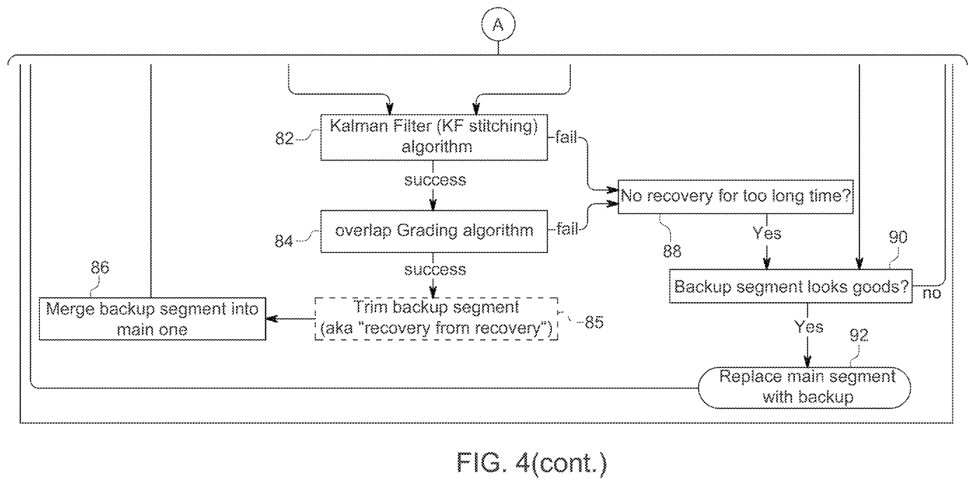

Reference is now made to FIG. 4, which depicts recovery stitching 32 in more detail, in accordance with some applications of the present invention. There may be cases where an incoming scan (step 24), along with its corresponding IMU data (step 42) and MTD data (step 38), cannot be correctly stitched to seed 34 of the current segment 27. For example, if the dentist performing the intraoral scan moves the scanner to a different area of the intraoral cavity or moves the scanner too quickly then the incoming scans may not be able to be stitched to the seed of the current segment. Recovery stitching component (step 32) of immediate stitching algorithm 20 handles cases where the ICP stitching (step 40) from the chain stitching component (step 26) fails to pass overlap grading (step 50) and/or motion grading (step 52) validators and thus is rejected from being stitched to seed 34 of the current segment.

If an incoming scan fails to stitch to seed 34 of the current (“main”) segment (step 72), instead of losing the data, the recovery stitching component keeps a “backup segment” 74. When a scan fails to stitch to seed 34 of “main” segment 27, recovery stitching component (step 32) attempts to stitch the scan to a seed 73 of backup segment 74 using the same chain stitching component (step 26) of the immediate stitching algorithm 20 as described hereinabove. When used in the recovery stitching, the chain stitching is referred to herein as step 26′. The backup segment is initiated using the same start-segment component 28 of the immediate stitching algorithm as described hereinabove. When used in the recovery stitching, the start segment is referred to as step 28′. For example, the first few scans sent to recovery stitching 32 become candidate seeds for backup segment 74 and as scans that fail to be stitched to seed 34 of main segment 27 are sent to backup segment 74, whichever candidate seed in the recovery stitching grows successfully becomes the start of backup segment 74. Overlap grading (step 50) and motion grading (step 52) are also used to verify the ICP stitching to the seed 73 of backup segment 74.

For some applications, the inability to stitch an incoming scan (step 24), along with its corresponding IMU data (step 42) and MTD data (step 38) to seed 34 of main segment 27 may be due to (a) a sporadic movement of the intraoral scanner that causes the stitching to fail overlap grading (step 50) and/or motion grading (step 52) verifiers or (b) the scanner being moved and now scanning a different region of the intraoral 3D surface. In both cases (a) and (b), processing device 22 continues to attempt to stitch new incoming scans to seed 34 of main segment 27. In the case of a sporadic movement of the intraoral scanner it is likely that subsequent consecutive incoming scans will stitch successfully to seed 34 of main segment 27, in which case the scan that was sent to the recovery stitching is discarded and backup segment 74 is reset (step 76). In the case where the scanner is now scanning a different region of the intraoral 3D surface, it is reasonable that subsequent consecutive incoming scans will also fail to stitch to seed 34 of main segment 27. Thus, backup segment 74 grows in parallel as the consecutive scans that failed to stitch seed 34 of main segment 27 are stitched to seed 73 of backup segment 74 (as described hereinabove).

As described hereinabove, the ICP stitching in the chain stitching component of the immediate stitching algorithm only stitches to seed 34 of current segment 27, i.e., the trailing subset of recent points added to the segment. Therefore, it is reasonable that although the scans that are sent to backup segment 74 failed to stitch to seed 34 of main segment 27, there may still be some overlap between backup segment 74 and some part of main segment 27 other than seed 34. Thus, recovery stitching component (step 32) of immediate stitching algorithm 20 attempts to stitch backup segment 74 to main segment 27 using a Kalman filter stitching algorithm (step 82) referred to herein as “KF stitching.” For the KF stitching (step 82), a target 3D point cloud is computed from the entire main segment 27 and a source 3D point cloud is computed from backup segment 74.

The KF stitching (step 82) is based on two concepts. The first concept is to split the main segment, i.e., the target 3D point cloud, into a plurality of sections and try to use a modified ICP between the backup segment (i.e., the source 3D point cloud computed from the backup segment) and the plurality of sections of the main segment (i.e., the target 3D point cloud) with different initial guesses (step 78). The modified ICP stitching (step 78) in the KF stitching algorithm (step 82) uses many iterations each with a small sample of points, e.g., less than 30 points, e.g., less than 20 points, e.g., 18 points. Each iteration, the target 3D point cloud position is modified with the Kalman filter state defining the position and rotation of the target cloud. The target cloud points are arranged in buckets according to their normals. The buckets are randomly sampled and the points from each bucket are also randomly sampled, thus giving uniformly distributed normals for each sample. Every few iterations, e.g., every 8 iterations, the overlap of the source cloud (i.e., the backup segment) and the target cloud (i.e., the main segment) is measured (step 84) using a sample of approximately 2000 points. If the current overlap is measured to be better than the previous overlap (from a few iterations ago) then the KF stitching between the backup segment and the main segment continues. If the overlap is not better than the previous overlap then the Kalman filter state is reset and the modified ICP starts again from a different place in the main segment.

As described hereinabove, for some applications, processing device 22 receives, during the intraoral scanning, a plurality of 2D images of the intraoral 3D surface captured under broadband illumination. The second concept in the KF stitching algorithm is to look for similar images (using image hashes) in the backup segment and the main segment and attempt to stitch scans that are near each other (step 80). That is, processing device 22 looks for 2D images of the intraoral 3D surface corresponding to backup segment 74 that are similar to 2D images of the intraoral 3D surface corresponding to main segment 27. For some applications, these image similarities are used by processing device 22 as a useful initial guess for the modified ICP (step 78) described hereinabove with respect to the first concept in the KF stitching (step 82).

If the KF stitching succeeds then the backup segment is joined to the main segment (step 86). For some applications, the backup segment may have accumulated a large number of scans such that when the backup segment is joined to the main segment it may result in a sudden influx of scans that need to be processed downstream and the dentist using the intraoral scanner may suddenly see a large part of the 3D model seeming to appear from nowhere on the display. Therefore, for some applications, a “recovery from recovery” logic is implemented to trim the backup segment into a reasonably small batch of scans before it is merged with the main segment (optional step 85). This improves the downstream processing and the user experiences only a short lag in the scanning and then the stitching appears to resume.

It is possible, however, that the KF stitching will not be able to stitch together the backup segment and the main segment. For example, there could be no overlap between the main segment and the backup segment if the scanner has been moved to an entirely different region in the intraoral cavity. In order to avoid the user-experienced time lag from being too long, there is a time limit placed on the recovery stitching (decision rectangle 88). If after a certain threshold period of time, e.g., a few seconds, e.g., 1 second, the backup segment is not rejoined to the main segment (answer to decision rectangle 88 is “yes,” i.e., the user does not see the stitching resume on the display) then one of the following options is implemented (based on decision rectangle 90):
i. If backup stitching is successfully growing (answer to decision rectangle 90 is “yes”) then backup segment 74 is simply redesignated as the new main segment 27′ (step 92) and the chain stitching component 26 of the immediate stitching algorithm 20 stops attempting to add scans to the old main segment 27. In this case the old main segment 27 is saved and the user sees the 3D image start to build the new area of the intraoral 3D surface now being scanned. Main segments 27 that are stopped and saved are eventually stitched together to form a 3D image of the entire scanned intraoral 3D surface.
ii. It is also possible that the intraoral scanner is moved to a new location even after the backup segment has started to grow, in which case the ICP stitching 26′ to seed 73 of backup segment 74 may also be repetitively failing the overlap grading (step 50) and motion grading (step 52) verifiers as new incoming scans fail to stich to either seed 34 of current main segment 27 or seed 73 of current backup segment 74. Thus, if the backup chain stitching and the main chain stitching appear stuck (answer to decision rectangle 90 is “No”), then backup segment 74 is reset (step 94) using the start segment component 28′, as described hereinabove.

Thus, as long as the dentist using the intraoral scanner keeps the scanner hovering over a particular region of the intraoral 3D surface (assuming the region is a scannable region, e.g., not hovering over moving tissue such as the patient's tongue), the recovery stitching component of the immediate stitching algorithm typically causes the scanning to resume within one or several seconds without losing the data accumulated during the time lag.

An example workflow of recovery stitching (step 32), performed by processing device 22, is as follows:
(a) receiving a first incoming scan by the intraoral scanner (including steps 24, 42, and 38), and attempting to register the first incoming scan (step 26) to registration seed 34,
(b) if the registration of the first incoming scan to the registration seed 34 fails (step 72), storing the first incoming scan as a scan in a backup candidate set of one or more intraoral scans (included in backup start segment step 28′),
(c) receiving a second incoming scan by the intraoral scanner (including steps 24, 42, and 38) and attempting to register the second incoming scan (step 26) to registration seed 34, and
(d):
(i) if the registration of the second incoming scan (step 26) to registration seed 34 fails, attempting to register the second incoming scan to the backup candidate set (included in recovery stitching start segment step 28′),
   (1) if the attempted registration to the backup candidate set is successful, updating the backup candidate set, and
   (2) if the attempted registration to the backup candidate set is unsuccessful, storing each of the first incoming scan and the second incoming scan separately, as scans in respective first and second backup candidate sets of one or more intraoral scans, and
(ii) if the registration of the second incoming scan (step 26) to registration seed 34 is successful, discarding the backup candidate sets generated in step (b) (step 76).

For some applications, the example workflow may further include:
(e) receiving a next incoming intraoral scan (including steps 24, 42, and 38) and attempting to register the next incoming intraoral scan (step 26) to registration seed 34, and
(f):
(i) if the registration of the next incoming scan (step 26) to registration seed 34 fails, attempting to register the second incoming scan to each of the backup candidate sets generated in step (d) (included in recovery stitching start segment step 28′):
   (1) if the attempted registration of the next incoming scan to a backup candidate set is successful, updating the backup candidate set,
   (2) if no attempted registration of the next incoming scan to a backup candidate set is successful, storing the next incoming scan as a scan in a new backup candidate set of one or more intraoral scans, and
   (3) if no backup candidate sets were stored in step (d), storing the next incoming scan as a scan in a new backup candidate set of one or more intraoral scans, and
(ii) if the registration of the next incoming scan (step 26) to the registration seed is successful, discarding all stored backup candidate sets (step 76).

For some applications, the example workflow may further include:
(g) iteratively repeating steps (e) and (f) until a given backup candidate set grows large enough to be considered a backup segment 74 of the 3D model of the intraoral surface,
(h) receiving a next incoming intraoral scan (including steps 24, 42, and 38) and attempting to register the next incoming intraoral scan (step 26) to registration seed 34 of main segment 27, and
(i):
(A) if the registration of the next incoming scan (step 26) to registration seed 34 of main segment 27 fails, attempting to register the next incoming scan (step 26′) to backup registration seed 73 of backup segment 74, wherein backup registration seed 73 is a subset of intraoral scans in backup segment 74 that immediately precede each next incoming scan, wherein:
   (1) if the attempted registration of the next incoming scan (step 26′) to backup registration seed 73 is successful, updating backup segment 74 of the 3D model, and
   (2) (a) if the attempted registration of the next incoming scan (step 26′) to backup registration seed 73 fails, increasing a counter of failed attempts to register a next incoming scan to the backup registration seed (step 96), and (b) if the counter exceeds a threshold number (step 97), discarding the backup segment (step 94) and storing the next incoming scan as a scan in a new backup candidate set of one or more intraoral scans, and
(B) if the registration of the next incoming scan (step 26) to registration seed 34 of main segment 27 is successful, discarding the backup segment (step 76).

For some applications, iteratively repeating steps (e) and (f) until a given backup candidate set grows large enough to be considered a backup segment 74 of the 3D model of the intraoral surface further includes attempting to register the backup segment of the 3D model to the main segment of the 3D model, e.g., using a Kalman filter algorithm (step 82).

For some applications:
(I) if the attempted registration of the backup segment of the 3D model to the main segment of the 3D model is successful, merging the backup segment of the 3D model with the main segment of the 3D model (step 86), outputting a view of the merged 3D model of the intraoral surface to the display, and subsequently using backup registration seed 73 of backup segment 74 as registration seed 34 of the merged 3D model for future income scans, and
(II) if the attempted registration of the backup segment of the 3D model to the main segment of the 3D model fails, waiting for the earlier of: (1) the backup segment to be updated, in which case the workflow further includes attempting to register the updated backup segment to the main segment, or (2) a new backup candidate set of one or more intraoral scans to grow large enough to be considered a new backup segment, in which case the workflow further includes attempting to register the new backup segment to the main segment.

For some applications, if upon performing step II the attempted registration of the updated backup segment to the main segment or of the new backup segment to the main segment fails, the method further comprises iteratively repeating the waiting of step (II), wherein if no registration of any backup segment to the main segment is successful for a threshold amount of time (decision rectangle 90), the workflow further includes storing main segment 27 of the 3D model as a first part of the 3D model of the intraoral surface and using the last backup segment 74 stored as the beginning of a new main segment 27′ for a second part of the 3D model of the intraoral surface (step 92).

Applications of the disclosure described herein can take the form of a computer program product accessible from a computer-usable or computer-readable medium (e.g., a non-transitory computer-readable medium) providing program code for use by or in connection with a computer or any instruction execution system, such as the processing device disclosed hereinabove. For the purpose of this description, a computer-usable or computer readable medium can be any apparatus that can comprise, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Typically, the computer-usable or computer readable medium is a non-transitory computer-usable or computer readable medium.

Examples of a computer-readable medium include a semiconductor or solid-state memory, magnetic tape, a removable computer diskette, a random-access memory (RAM), a read-only memory (ROM), a rigid magnetic disk and an optical disk. Current examples of optical disks include compact disk-read only memory (CD-ROM), compact disk-read/write (CD-R/W) and DVD. For some applications, cloud storage, and/or storage in a remote server is used.

A data processing system suitable for storing and/or executing program code will include at least one processor (e.g., processing device 22 disclosed hereinabove) coupled directly or indirectly to memory elements through a system bus. The memory elements can include local memory employed during actual execution of the program code, bulk storage, and cache memories which provide temporary storage of at least some program code in order to reduce the number of times code must be retrieved from bulk storage during execution. The system can read the inventive instructions on the program storage devices and follow these instructions to execute the methodology of the embodiments of the disclosure.

Network adapters may be coupled to the processor to enable the processor to become coupled to other processors or remote printers or storage devices through intervening private or public networks. Modems, cable modem and Ethernet cards are just a few of the currently available types of network adapters.

Computer program code for carrying out operations of the present disclosure may be written in any combination of one or more programming languages, including an object-oriented programming language such as Java, Smalltalk, C++, Python, or the like and conventional procedural programming languages, such as the C programming language or similar programming languages.

It will be understood that the methods described herein can be implemented by computer program instructions. These computer program instructions may be provided to a processor of a general-purpose computer, special purpose computer, or other programmable data processing apparatus to produce a machine, such that the instructions, which execute via the processor of the computer (e.g., processing device 22 disclosed hereinabove) or other programmable data processing apparatus, create means for implementing the functions/acts specified in the methods described in the present application. These computer program instructions may also be stored in a computer-readable medium (e.g., a non-transitory computer-readable medium) that can direct a computer or other programmable data processing apparatus to function in a particular manner, such that the instructions stored in the computer-readable medium produce an article of manufacture including instruction means which implement the function/act specified in the methods described in the present application. The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide processes for implementing the functions/acts specified in the methods described in the present application.

The processing device disclosed hereinabove is typically a hardware device programmed with computer program instructions to produce a special purpose computer. For example, when programmed to perform the methods described herein, the processing device typically acts as a special purpose processing device. Typically, the operations described herein that are performed by computer processors transform the physical state of a memory, which is a real physical article, to have a different magnetic polarity, electrical charge, or the like depending on the technology of the memory that is used.

It will be appreciated by persons skilled in the art that the present disclosure is not limited to what has been particularly shown and described hereinabove. Rather, the scope of the present disclosure includes both combinations and subcombinations of the various features described hereinabove, as well as variations and modifications thereof that are not in the prior art, which would occur to persons skilled in the art upon reading the foregoing description.

What is claimed is:

1. An intraoral scanning system, comprising:
an intraoral scanner to generate a plurality of intraoral scans of an intraoral three-dimensional (3D) surface during intraoral scanning of the intraoral 3D surface, each intraoral scan of the plurality of intraoral scans comprising an image of a distribution of features of light projected on the intraoral 3D surface; and
at least one computer processor configured to:
receive, during the intraoral scanning of the intraoral 3D surface by the intraoral scanner, the plurality of intraoral scans of the intraoral 3D surface generated by the intraoral scanner;
register the plurality of intraoral scans to each other during the intraoral scanning to update a 3D model of the intraoral 3D surface, wherein the 3D model is updated at a rate of 3-100 times per second; and
output a view of the 3D model of the intraoral 3D surface to a display.

2. The intraoral scanning system according to claim 1, wherein each intraoral scan of the plurality of intraoral scans comprises an image of a distribution of 400-3000 discrete unconnected spots of light projected on the intraoral 3D surface.

3. An intraoral scanning system, comprising:
an intraoral scanner to generate a plurality of intraoral scans of an intraoral three-dimensional (3D) surface during intraoral scanning of the intraoral 3D surface, each intraoral scan of the plurality of consecutive intraoral scans comprising an image of a distribution of discrete unconnected spots of light projected on the intraoral 3D surface; and
at least one computer processor configured to:
receive, during the intraoral scanning of the intraoral 3D surface by the intraoral scanner, the plurality of consecutive intraoral scans of the intraoral 3D surface generated by the intraoral scanner;
register the plurality of intraoral scans to each other during the intraoral scanning to update a segment of a 3D model of the intraoral 3D surface by registering each incoming scan of a subset of the plurality of intraoral scans to a registration seed, wherein the registration seed is a subset of the plurality of intraoral scans that immediately precede the incoming scan and that have previously been registered together; and
output a view of the segment of the 3D model of the intraoral 3D surface to a display.

4. The intraoral scanning system according to claim 3, wherein the segment of the 3D model is updated at a rate of 3-100 times per second.

5. The intraoral scanning system according to claim 3, wherein the registration seed comprises a subset of at last 2-8 intraoral scans generated by the intraoral scanner during the intraoral scanning.

6. The intraoral scanning system according to claim 3, wherein the at least one computer processor is further configured to select a quantity of intraoral scans to be included in the registration seed such that the registration seed comprises images of a combined total of 500-5000 discrete unconnected spots of light projected on the intraoral 3D surface.

7. The intraoral scanning system according to claim 3, wherein the at least one computer processor is further configured to:
compute a source 3D point cloud from the distribution of discrete unconnected spots of light captured in each incoming scan and compute a target 3D point cloud from the registration seed, wherein registering each incoming scan of the subset of the plurality of intraoral scans to the registration seed comprises using an Iterative Closest Point (ICP) algorithm to register the source 3D point cloud to the target 3D point cloud,
receive, during the intraoral scanning, a plurality of 2D images of the intraoral 3D surface captured under broadband illumination,
pair one or more of the plurality of 2D images with the incoming scan, and based on the pairing, classify a subset of the points in the source 3D point cloud as corresponding to a spot of light projected on rigid tissue, and
use as an input to the ICP algorithm only the points in the source 3D point cloud that are classified as corresponding to a spot of light projected on rigid tissue.

8. The intraoral scanning system according to claim 3, wherein the at least one computer processor is further configured to:
compute a source 3D point cloud from the distribution of discrete unconnected spots of light captured in each incoming scan and compute a target 3D point cloud from the registration seed, wherein registering each incoming scan of the subset of the plurality of intraoral scans to the registration seed comprises using an Iterative Closest Point (ICP) algorithm to register the source 3D point cloud to the target 3D point cloud,
receive, during the intraoral scanning, data pertaining to motion of the intraoral scanner from an inertial measurement unit (IMU) disposed within the intraoral scanner,
for each incoming scan of the subset of the plurality of intraoral scans, use a filter to predict an expected position of the intraoral scanner by extrapolating a previous trajectory of the motion of the intraoral scanner using the data from the IMU, and
provide the ICP algorithm with an initial estimate based on (i) the expected position predicted by the filter, and (ii) an intraoral scan that immediately preceded the incoming scan.

9. The intraoral scanning system according to claim 3, wherein the at least one computer processor is further configured to:
compute a source 3D point cloud from the distribution of discrete unconnected spots of light captured in each incoming scan and compute a target 3D point cloud from the registration seed, wherein registering each incoming scan of the subset of the plurality of intraoral scans to the registration seed comprises using an Iterative Closest Point (ICP) algorithm to register the source 3D point cloud to the target 3D point cloud,
attempt to compute a normal for each point in the source 3D point cloud, and
use as an input to the ICP algorithm only the points in the source 3D point cloud for which the normal was successfully computed.

10. The intraoral scanning system according to claim 3, wherein the at least one computer processor is further configured to:
compute a source 3D point cloud from the distribution of discrete unconnected spots of light captured in each incoming scan and computing a target 3D point cloud from the registration seed, wherein registering each incoming scan of the subset of the plurality of intraoral scans to the registration seed comprises using an Iterative Closest Point (ICP) algorithm to register the source 3D point cloud to the target 3D point cloud, and
subsequently to the ICP algorithm registering the source 3D point cloud to the target 3D point cloud, assess an extent of overlap between the source 3D point cloud and the target 3D point cloud.

11. The intraoral scanning system according to claim 10, wherein assessing the extent of overlap comprises:
outputting a subset of pairs of matching points between the source 3D point cloud and the target 3D point cloud, each pair of points determined to be matching points by the ICP algorithm,
for each pair of matching points, extracting a plurality of features,
inputting the extracted plurality of features into a classifier algorithm,
receiving from the classifier algorithm an overlap-grading-score relating to the extent of overlap between the source 3D point cloud and the target 3D point cloud, and
rejecting the registration of the incoming scan by the ICP algorithm in response to the overlap-grading-score being lower than a threshold score.

12. The intraoral scanning system according to claim 3, wherein the at least one computer processor is further configured to:
compute a source 3D point cloud from the distribution of discrete unconnected spots of light captured in each incoming scan and compute a target 3D point cloud from the registration seed, wherein registering each incoming scan of the subset of the plurality of intraoral scans to the registration seed comprises using an Iterative Closest Point (ICP) algorithm to register the source 3D point cloud to the target 3D point cloud,
receive, during the intraoral scanning, data pertaining to motion of the intraoral scanner from an inertial measurement unit (IMU) disposed within the intraoral scanner,
assess the registration by the ICP algorithm of the source 3D point cloud to the target 3D point cloud by comparing the registration to data from the IMU, and
reject the registration of the incoming scan by the ICP algorithm in response to the registration of the source 3D point cloud to the target 3D point cloud contradicting the data from the IMU.

13. The intraoral scanning system according to claim 3, wherein the at least one computer processor is further configured to:
when no registration seed is available:
(a) receive a first incoming scan by the intraoral scanner,
(b) receive a second incoming scan by the intraoral scanner,
(c) attempt to register the second incoming scan to the first incoming scan,
(d):
(i) if the attempted registration of the second incoming scan to the first incoming scan is successful, store a set of the first and second incoming scans that have been registered to each other as a candidate set for the registration seed, and
(ii) if the attempted registration of the second incoming scan to the first incoming scan is unsuccessful, store each of the first and second incoming scans separately as candidate sets for the registration seed,
(e) receive a next incoming scan by the intraoral scanner and attempt to register the next incoming scan to each of the stored candidate sets generated in step (d),
(f):
(i) if the attempted registration of the next incoming scan to a candidate set is successful, update the candidate set, and
(ii) if no attempted registration of the next incoming scan to a candidate set is successful, store the next incoming scan as a new candidate set for the registration seed, and
(g) iteratively repeat steps (e) and (f) until at least one candidate set is large enough to be used as the registration seed.

14. The intraoral scanning system according to claim 13, wherein the at least one computer processor is further configured to discard an oldest candidate set when a number of stored candidate sets surpasses a threshold number.

15. The intraoral scanning system according to claim 3, wherein the at least one computer processor is further configured to:
(a) receive a first incoming scan by the intraoral scanner and attempt to register the first incoming scan to the registration seed,
(b) if the registration of the first incoming scan to the registration seed fails, store the first incoming scan as a scan in a backup candidate set of one or more intraoral scans,
(c) receive a second incoming scan by the intraoral scanner and attempt to register the second incoming scan to registration seed, and
(d):
(i) if the registration of the second incoming scan to the registration seed fails, attempt to register the second incoming scan to the backup candidate set,
(1) if the attempted registration to the backup candidate set is successful, update the backup candidate set, and
(2) if the attempted registration to the backup candidate set is unsuccessful, store each of the first incoming scan and the second incoming scan separately, as scans in respective first and second backup candidate sets of one or more intraoral scans, and
(ii) if the registration of the second incoming scan to the registration seed is successful, discard the backup candidate sets generated in step (b).

16. The intraoral scanning system according to claim 15, wherein the at least one computer processor is further configured to:
(e) receive a next incoming intraoral scan and attempt to register the next incoming intraoral scan to the registration seed, and
(f):
(i) if the registration of the next incoming scan to the registration seed fails, attempt to register the second incoming scan to each of the backup candidate sets generated in step (d):
(1) if the attempted registration of the next incoming scan to a backup candidate set is successful, update the backup candidate set,
(2) if no attempted registration of the next incoming scan to a backup candidate set is successful, store the next incoming scan as a scan in a new backup candidate set of one or more intraoral scans, and
(3) if no backup candidate sets were stored in step (d), store the next incoming scan as a scan in a new backup candidate set of one or more intraoral scans, and
(ii) if the registration of the next incoming scan to the registration seed is successful, discard all stored backup candidate sets.

17. The intraoral scanning system according to claim 16, wherein the segment of the 3D model is a main segment of the 3D model, and wherein the at least one computer processor is further configured to:
(g) iteratively repeat steps (e) and (f) until a given backup candidate set grows large enough to be considered a backup segment of the 3D model of the intraoral 3D surface,
(h) receive a next incoming intraoral scan and attempt to register the next incoming intraoral scan to the registration seed of the main segment, and
(i):
(A) if the registration of the next incoming scan to the registration seed of the main segment fails, attempt to register the next incoming scan to a backup registration seed of the backup segment, wherein the backup registration seed is a subset of intraoral scans in the backup segment that immediately precede each next incoming scan, wherein:
(1) if the attempted registration of the next incoming scan to the backup registration seed is successful, update the backup segment of the 3D model, and
(2) (a) if the attempted registration of the next incoming scan to the backup registration seed fails, increase a counter of failed attempts to register a next incoming scan to the backup registration seed, and (b) if the counter exceeds a threshold number, discard the backup segment and store the next incoming scan as a scan in a new backup candidate set of one or more intraoral scans, and
(B) if the registration of the next incoming scan to the registration seed of the main segment is successful, discard the backup segment.

18. An intraoral scanning system, comprising:
an intraoral scanner to generate a plurality of intraoral scans of an intraoral three-dimensional (3D) surface during intraoral scanning of the intraoral 3D surface, each intraoral scan of the plurality of intraoral scans comprising an image of a distribution of features of light projected on the intraoral 3D surface; and
at least one computer processor configured to:
(a) receive a first incoming scan of the plurality of intraoral scans,
(b) receive a second incoming scan by the plurality of intraoral scans,
(c) attempt to register the second incoming scan to the first incoming scan,
(d):
(i) if the attempted registration of the second incoming scan to the first incoming scan is successful, store a set of the first and second incoming scans that have been registered to each other as a candidate set for a registration seed, and
(ii) if the attempted registration of the second incoming scan to the first incoming scan is unsuccessful, store each of the first and second incoming scans separately as candidate sets for the registration seed,
(e) receive a next incoming scan of the plurality of intraoral scans and attempt to register the next incoming scan to each of the stored candidate sets generated in step (d),
(f):
(i) if the attempted registration of the next incoming scan to a candidate set is successful, update the candidate set, and
(ii) if no attempted registration of the next incoming scan to a candidate set is successful, store the next incoming scan as a new candidate set for the registration seed, and
(g) iteratively repeat steps (e) and (f) until at least one candidate set is large enough to be used as the registration seed,
receive a remainder of the plurality of intraoral scans;
generate a 3D model of the intraoral 3D surface using the registration seed and one or more additional intraoral scans of the plurality of intraoral scans, and
output a view of the 3D model of the intraoral 3D surface to a display.

19. The intraoral scanning system of claim 18, wherein using the registration seed comprises registering the one or more additional intraoral scans to the registration seed.

20. The intraoral scanning system of claim 19, wherein the at least one computer processor is further configured to:
compute a source 3D point cloud from the distribution of features of light captured in the one or more additional intraoral scans, and
compute a target 3D point cloud from the registration seed,
wherein registering the one or more additional intraoral scans to the registration seed comprises using an Iterative Closest Point (ICP) algorithm to register the source 3D point cloud to the target 3D point cloud.
